(19) United States
(12) Patent Application Publication
(10) Pub. No.: US 2023/0409778 A1
(43) Pub. Date: Dec. 21, 2023

(54) ALGORITHMS FOR PREDICTING VUG CONNECTIVITY

(71) Applicant: University of Kansas, Lawrence, KS (US)

(72) Inventors: Hassan Eltom, Lawrence, KS (US); Eugene C. Rankey, Lawrence, KS (US); Robert H. Goldstein, Lawrence, KS (US); Reza Barati, Lawrence, KS (US); Stephen Hasiotis, Lawrence, KS (US)

(21) Appl. No.: 18/253,520

(22) PCT Filed: Dec. 14, 2021
(86) PCT No.: PCT/US2021/063329

§ 371 Date: May 18, 2023

Related U.S. Application Data
(60) Provisional application No. 63/125,314, filed on Dec. 14, 2020.

(51) Int. Cl. G06F 30/20

(52) U.S. Cl. CPC G06F 30/20

(57) ABSTRACT

Accurately predicting permeability of a material such as a rocks of a subsurface fluid reservoir is challenging. An accurate permeability prediction can be generated by receiving input data from a plurality of data sources and deriving vug attribute parameters based, at least in part, on the input data. The vug attribute parameters may be provided to a model configured to generate modelled rock property data. The modelled rock property data may include a probability indicative of vug connectedness of vugs located in rocks of the subsurface fluid reservoir. The probability of vug connectedness may be indicative of a permeability probability of rocks of the subsurface fluid reservoir.

### CROSS-REFERENCE TO RELATED APPLICATION

The present application claims the benefit of priority from U.S. Provisional Patent Application No. 63/125,314 filed Dec. 14, 2020 and entitled “VUG CONNECTIVITY PREDICTION DEVICE,” the disclosure of which is incorporated by reference herein in its entirety.

### TECHNICAL FIELD

The present invention relates generally to modelling rock formations and more specifically to systems and methods for modelling and predicting permeability of rock formations.

### BACKGROUND

Characterizing permeability of rock formations is difficult. The permeability of rock formations may depend on the presence of openings in the rock formation. One example of such an opening is a vug, which can vary in size, shape, and abundance. Vugs have been broadly defined as nondescript-shape, non-fabric selective pores, commonly of solution origin. While this definition considers the importance of diagenesis in making vugs, it lacks a clear application to petrophysical properties. Another characterization of vugs, which is more applicable to their petrophysical properties, divides carbonate pores into two classes: (1) pore space located between grains or crystals, termed interparticle or intercrystalline pores, respectively; and (2) all other pore space, called vuggy pores. Under the second part of this characterization, vugs may be further grouped into two general classes: separate vugs and touching vugs, where separate vugs are isolated and interconnected only through matrix porosity and touching vugs refer to vugs that connect to other vugs and through matrix porosity. These distinct styles of interconnectivity imply that simply increasing the volume or number of vugs alone does not necessarily increase permeability, whereas increasing the abundance of touching vugs could increase permeability greatly.

The performance of a vuggy reservoir, therefore, may be impacted by the lateral and vertical nature of vug-to-vug connections, and the character of the non-vuggy matrix porosity in the reservoir. However, it is difficult to characterize or predict the performance of a reservoir based on the mere presence of vugs (e.g., separate vugs, isolated vugs, or both). In particular, vugs can impact production in different ways. In some cases, vugs enhance production, but in others, vugs inhibit full recovery. For example, carbonate reservoirs in Ordovician strata of the Tarim Basin, China, contain two distinct zones, one with touching vugs and another with separate vugs. The touching vugs in these strata reflect prominent and connected pores produced by dissolution of grainstone, packstone, and rudstone facies, whereas the separate vugs result from dissolution within dolomites, and are connected only through intercrystalline pores of the matrix. As a result, the permeability of the reservoir zone with touching vugs is markedly higher than the permeability of the reservoir zone with separate vugs. A contrasting example of how vugs can influence the performance of a reservoir is illustrated in the negative impact of high-permeability (super-k) zones in the Jurassic Arab-D reservoir, Saudi Arabia. These zones consist of porous dolomite and solution-enlarged skeletal molds (e.g., vugs) forming a system of touching vugs connected by a permeable matrix. In super-k zones, the total vug volume and pattern of the vug connectivity creates high permeability pathways, and focuses flow. These preferred flow units lead to inefficient sweep that bypasses much of the reservoir and leads to early water breakthrough.

As shown by these examples, it can be difficult to characterize the impact of vugs on a reservoir. One reason why it is difficult to characterize the impact of vugs on a reservoir is that existing tools are limited. For example, vugs may be characterized based on data collected from samples extracted from rock formations, or from well logs. The samples usually are either core plugs or whole cores. Core plugs inadequately sample vugs, thereby resulting in erroneous permeability predictions of rock formations from which the core plugs were extracted. Whole cores and well logs may provide more information about the vugs within a rock formation but are insufficient for identifying and characterizing vug networks (e.g., a series of connected vugs). Accordingly, currently available tools and techniques for characterizing rock formation permeability may lead to erroneous permeability predictions and an incorrect understanding of how vugs present in a rock formation will impact performance of a reservoir.

### SUMMARY

Systems, devices, and methods for predicting permeability of a material are disclosed herein. Data associated with one or more characteristics of the material may be compiled. For instance, the material may be rocks of a rock formation. The data associated with one or more characteristics of the material may include nuclear magnetic resonance (NMR) logs, image logs, computerized tomography (CT) scans, sonic scans, and/or other data indicative of one more physical and/or chemical properties of the rock. The data may be collected from one or more data sources. The one or more data sources may include sensors disposed in or around the rock formation, computer databases including physical and/or chemical data about rocks of the rock formation, and/or autonomous devices configured to obtain and compile physical and/or chemical data about rocks of the rock formation. Vug attribute parameters may be derived from the data. Vug attribute parameters derived from the data may include, as non-limiting examples, a mean quantity (e.g., abundance) of vugs in the rock formation, a mean size (e.g., diameter) of vugs in the rock formation, a representative shape of vugs of the rock formation, and/or an aspect ratio of dimensions of a representative vug shape of the vugs present in the rock formation. In an implementation, vug attribute parameters derived from the data may be used to generate a vug network model. A modelling engine may be executed against the vug attribute parameters to generate modelled rock property data indicative of a predicted permeability of the material. The modelled rock property data may be displayed on a graphical user interface (GUI). The vug network model may be an ergodic model. The vug network model may indicate a probability that vugs of a rock formation have particular physical characteristics such as an extent of vug connectedness indicative of a permeability of the rock, which may provide insights into the permeability of the modeled rock formation or other types of information.

Unlike existing methodologies, which fail to account for macro properties of the rock formation and thereby lead to erroneous understanding of formation permeability, embodiments of the present disclosure account for macro physical characteristics of a rock formation. Therefore, the disclosed systems and methods may provide significantly more accurate permeability predictions (e.g., vug connectedness predictions) than currently existing methodologies and tools. Moreover, the improved understanding of permeability provided by aspects of the present disclosure may provide insights into possible fluid flow properties through a vuggy reservoir.

Accordingly, in one aspect of the disclosure, a non-transitory computer-readable medium storing instructions is disclosed that, when executed by one or more processors, cause the one or more processors to perform operations for evaluating permeability of a material. The operations include compiling data associated with one or more characteristics of the material. The operations further include deriving vug attribute parameters from the data, executing a modelling engine against the vug attribute parameters, and predicting the permeability of the material based on the execution of the modelling engine against the vug attribute parameters. Further, the operations include displaying information associated with the permeability of the material at a graphical user interface (GUI).

In an additional aspect of the disclosure, a device for evaluating permeability of rock extracted from a rock formation is disclosed. The device includes a memory configured to store instructions and data associated with one or more characteristics of the rock. Additionally, the device includes a processor configured to execute instructions to derive vug attribute parameters from the data, generate a model of vugs comprising the rock formation based, at least in part, on the vug attribute parameters, and generate modelled rock property data that includes a probability indicative of vug connectedness based, at least in part, on the model.

In yet an additional aspect of the disclosure, a system for evaluating permeability of rock extracted from a rock formation and for predicting a type of fluid flow through the rock formation is disclosed. The system includes a first device configured to generate modelled rock property data comprising a probability that the rock is permeable, the device configured to generate the modelled rock property data based on vug attribute parameters derived from data corresponding to characteristics of the rock and of the rock formation. Additionally, the system includes at least one data source of a plurality of data sources, wherein the data source is configured to provide the data to the first device. Further, the system includes at least a second device configured to receive the modelled rock property data and to display the modelled rock property data.

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings, in which:

FIG. 1 is a block diagram illustrating an example material permeability prediction system according to embodiments of the present disclosure;

FIGS. 2A-2C depict vug network models according to embodiments of the present disclosure;

FIG. 3 depicts aspects of a permeability prediction process (e.g., to predict vug connectedness) according to embodiments of the present disclosure;

FIG. 4 depicts an example of probabilities of achieving different classes of permeability from networks of vugs with different attributes according to embodiments of the present disclosure;

FIGS. 5A and 5B depict additional examples of probabilities of achieving permeability from networks of vugs given different vug attributes according to embodiments of the present disclosure;

FIG. 6 describes a process for modelling permeability from vug attributes according to embodiments of the present disclosure;

FIG. 7 depicts correlation between fluid flow rate in a reservoir and predictions of material permeability;

FIGS. 8A and 8B depict particular experimental results showing control of vug shape and abundance on connectivity, according to embodiments of the present disclosure;

FIGS. 9A and 9B depict additional experimental results showing control of vug shape and abundance on connectivity, according to embodiments of the present disclosure;

FIG. 10 depicts and compares predictions generated by the model against empirical data as a validation of the method, according to embodiments of the present disclosure;

FIG. 11 shows validation results, according to embodiments of the present disclosure; and

FIG. 12 is a diagram illustrating exemplary sample data obtained from a rock formation in accordance with aspects of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

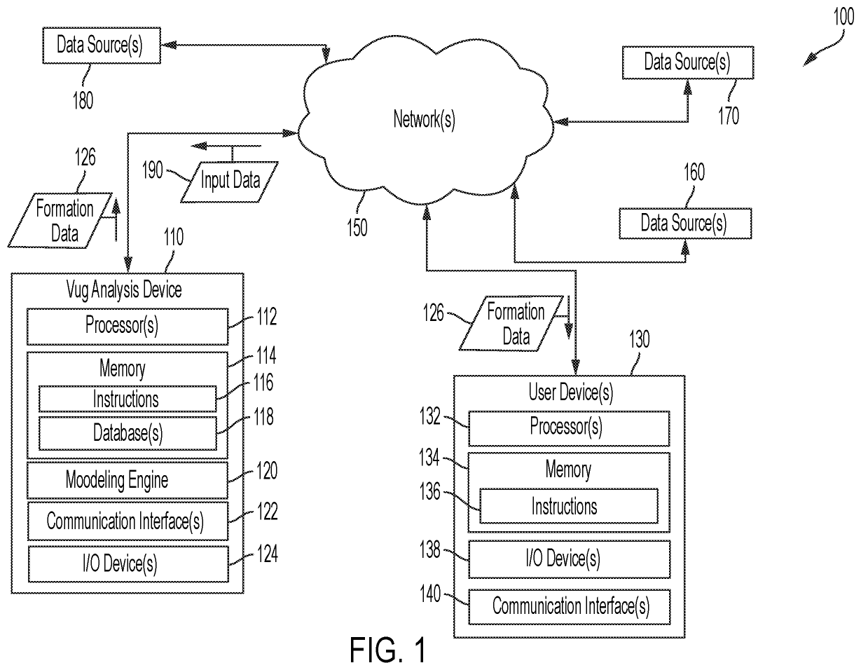

Referring to FIG. 1, a block diagram illustrating an example material permeability prediction system is shown as material permeability prediction system 100. Material permeability prediction system 100 includes vug analysis device 110, at least one user device 130, a plurality of data sources 160, 170, 180, and network 150. Although only one user device 130 is shown in FIG. 1, it is understood that material permeability prediction system 100 may include a plurality of user devices such as user device 130.

Vug analysis device 110 may be configured to generate vug connectedness predictions based on a set of input data, such as input data 190. Vug analysis device 110 may include one or more processors 112, memory 114, modelling engine 120, communication interface 122, and I/O device 124. Each of the one or more processors 112 may be a microprocessor, a graphical processing unit (GPU), one or more field programmable gate arrays (FPGAs), a microcontroller, and/or an application specific integrated circuit (ASIC) or other logic circuitry configured to perform the operations described herein with reference to vug analysis device 110.

Memory 114 may include a random access memory (RAM), which can be synchronous RAM (SRAM), dynamic RAM (DRAM), synchronous dynamic RAM (SDRAM), or the like. Memory 114 may also include read only memory (ROM), which can be programmable read only member (PROM), erasable programmable read only member (EPROM), electrically erasable programmable read only memory (EEPROM), optical storage, or the like. Additionally, memory 114 may include hard disk drives (HDDs), solid state disk drives (SSDs), and other memory devices configured to store data in a persistent or a non-persistent state. Memory 114 may be configured to store instructions 116 and database 118. Instructions 116 may be comprised of computer-readable code that, when executed by the one or more processors 112, cause the one or more processors 112 to perform the functionality described herein with respect to vug analysis device 110. Database 118 may be configured to store information. For example, database 118 may be configured to store input data 190 and/or modelled rock property data 126.

Processor 112 may be configured to store input data 190 in database 118 for further analysis. Additionally, processor 112 may be configured to execute modelling engine 120. In implementations, modelling engine 120 may be executable by the processor 112 to perform various functions, such as deriving vug attribute parameters based on input data 190, generating vug connectedness predictions based on vug attribute parameters, or other types of information described herein. In other implementations, modelling engine 120 may include a second processor and/or a special purpose processor (e.g., a GPU, ASIC, etc.) configured to implement instructions 116 to perform the functions described with reference to the modelling engine 120.

Modelling engine 120 may be configured to derive vug attribute parameters from input data 190. Additionally, modelling engine 120 may be configured to generate modelled rock property data 126 based on one or more vug attribute parameters. To illustrate, modelling engine 120 may be configured to perform a statistical analysis of vug attribute parameter data to derive a probability indicative of a permeability of a material under investigation (e.g., that rocks of a rock formation are permeable from connected vugs). The probability indicative of the permeability of the material under investigation may be a probability of vug connectedness (e.g., that vugs of a rock formation constitute interconnected vug passageways or that vugs of a rock formation are isolated vugs). The statistical analysis may include regression, Bayesian inference, and/or other statistical techniques. In implementations, modelling engine 120 may be configured to quickly (e.g., within seconds) generate predictions (e.g., vug connectedness predictions) based on a large volume (e.g., gigabytes, terabytes) of input data 190. In an aspect, modelling engine 120 may be executable by the one or more processors 112. Additionally or alternatively, modelling engine 120 may include at least one second processor (e.g., one or more microprocessors, GPUs, one or more FPGAs, microcontrollers, ASIC, or any combination thereof) configured to perform operations of the modelling engine 120 as described herein.

Communication interface 122 may be a network interface card (NIC), a transceiver, a transmitter, and/or a receiver. Communication interface 122 may be configured to communicate using a plurality of communication protocols, such as a Bluetooth™ protocol, a Zigbee™ protocol, and/or a cellular communication protocol, such as any of the 3G, 4G, and/or 5G communication protocols. Additionally, communication interface 122 may be any networking hardware capable of communicating using the 802.11 communication standard, the Ethernet communication standard, or other communication standards that may be developed.

The one or more input/output (I/O) devices 124 may include any device configured to receive input from a user (e.g., a user of vug analysis device 110) and/or to provide output to the user. As examples, the one or more I/O devices 124 may include a keyboard, a monitor or other display, a mouse, a printer, other types of I/O devices, or a combination thereof. In an implementation, processor 112 may be configured to render data to the one or more I/O devices 124, such to a display device, or read information from the one or more I/O devices 124, such as inputs provided via a keyboard, a mouse, etc.

As shown in FIG. 1, material permeability prediction system 100 includes a plurality of data sources 160, 170, and 180. Data source 160 may be a database storing well log data, image log data, core sample data, and/or other data associated with a material under investigation, such as known attributes of the rocks of a rock formation. The database may include nuclear magnetic resonance (NMR) spectroscopy of the rocks of the rock formation and/or computerized tomography (CT) images of the rocks of the rock formation. The database may include other diagenetic (chemical and/or physical) data corresponding to the rocks of the rock formation. The database may be stored on a computer accessible to vug analysis device 110 via network 150. Data source 170 may be a sensor disposed within a material under investigation, such as rocks of a rock formation. Such a sensor may include instruments to collect geophysical and/or geochemical data associated with the rocks of the rock formation. For instance, the sensor may include an NMR spectrometer and/or a CT scanner to generate NMR logs and CT images of rocks of the rock formation. Data source 180 may be an autonomous vehicle equipped with instruments to extract geophysical data about a material under investigation, such as rocks of a rock formation. For instance, the autonomous vehicle may be equipped with instruments configured to perform seismic analysis of rocks of the rock formation. The plurality of data sources 160, 170, 180, individually or in combination, may generate input data 190. Input data 190 may include physical and/or chemical data about a material under investigation, such as physical and/or chemical data about rocks of a rock formation. Such data may include NMR logs, CT images, seismographic studies, and/or other geophysical, textural, stratigraphic, and/or geochemical data.

Network 150 may be any of a wide area network (WAN) and/or a local area network (LAN). Network 150 may be the Internet. Network 150 may be configured to receive input data 190 from the plurality of data sources 160, 170, 180. Additionally network 150 may be configured to relay input data 190 to vug analysis device 110 and to relay modelled rock property data 126 from vug analysis device 110 to user device 130.

User device 130 may be configured to receive modelled rock property data 126 via network 150 from vug analysis device 110. In an implementation, user device 130 includes processor 132, memory 134, I/O device 138, and communication interface 140. Processor 132 may be configured to analyze modelled rock property data 126. Alternatively or additionally, processor 132 may be configured to cause I/O device 138 to render modelled rock property data 126. Processor 132 may be one or more microprocessors, a GPU, one or more FPGAs, a microcontroller, and/or an ASIC.

Memory 134 may be a RAM, which can be SRAM, DRAM, SDRAM, or the like. Memory 134 may also include ROM, which can be PROM, EPROM, EEPROM, optical storage, or the like. Memory 134 may be configured to store instructions 136 operable to executed by processor 132. Further, memory 134 may be configured to store modelled rock property data 126. Additionally, memory 134 may include HDDs, SSDs, and/or other memory devices configured to store data in a persistent or a non-persistent state.

The I/O device 138 may include any device configured to receive input from a user (e.g., a user of user device) and/or to provide output to the user. As examples, I/O device 138 may include a keyboard, a monitor or other display, a mouse, and/or a printer. In an implementation, processor 132 may be configured to render modelled rock property data 126 on I/O device 138, such as a display.

Communication interface 140 may be a network interface card (NIC), a transceiver, a transmitter, and/or a receiver. In implementations, communication interface 140 may be configured to receive modelled rock property data 126. For example, communication interface 140 may be configured to receive modelled rock property data 126 from network 150. Alternatively or additionally, communication interface 140 may be configured to receive modelled rock property data 126 from vug analysis device 110. Communication interface 140 may be configured to communicate using a plurality of communication protocols, such as a Bluetooth™ protocol, a Zigbee™ protocol, and/or a cellular communication protocol, such as any of the 3G, 4G, and/or 5G communication protocols. Additionally, communication interface 140 may be any networking hardware capable of communicating using the 802.11 communication standard, the Ethernet communication standard, or other communication standards that may be developed.

In an implementation, input data 190, generated by or stored in at least one data source of a plurality of data sources 160, 170, and 180, is received at communication interface 122 of vug analysis device 110 via network 150. In examples, vug analysis device 110 may receive input data 190 from one or more of data sources 160, 170, and 180 simultaneously or at different time instances.

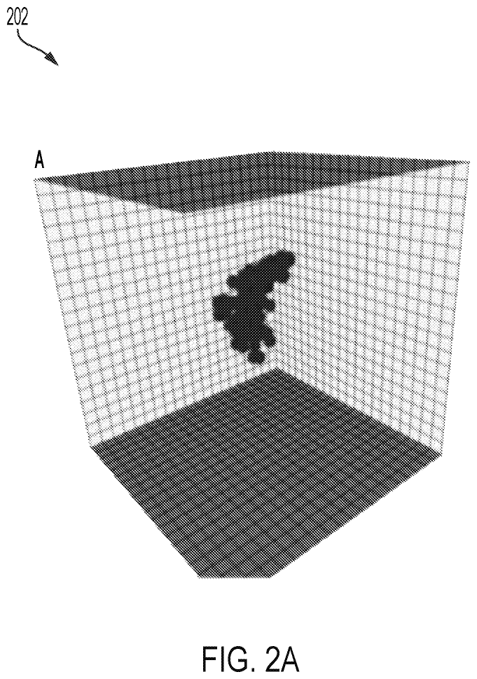

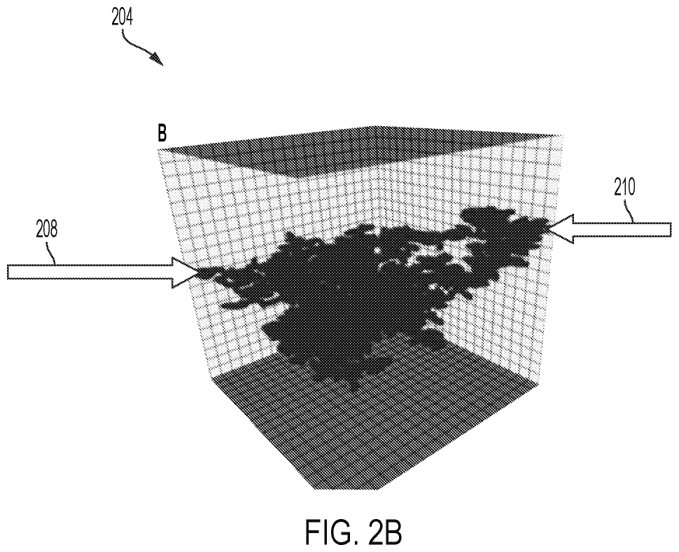

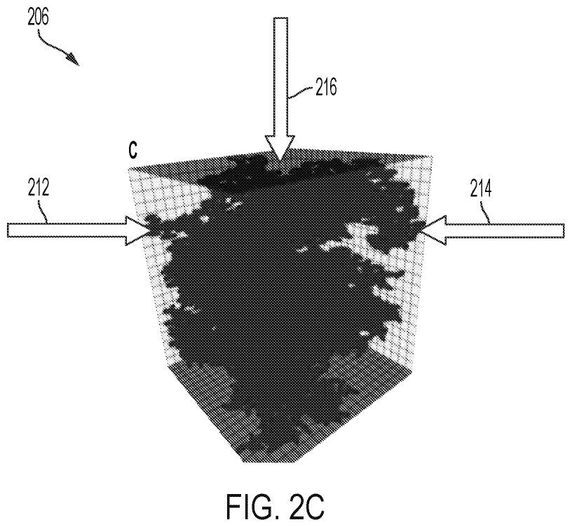

Modelling engine 120 may be configured to generate a model of a reservoir containing vugs. Vugs may be isolated or connected transversely or omnidirectionally. Isolated vugs generally are not connected with one another except via intercrystalline and interparticle pores of a rock matrix of rock layers in which the vugs are disposed. By contrast, transverse vug passageways and omnidirectional vug passageways exhibit vug interconnectedness (i.e., vugs comprising the vug networks are generally connected to one another). FIG. 2A depicts isolated vugs, FIG. 2B depicts transversely connected vugs, and FIG. 2C depicts omnidirectionally connected vugs. As shown in FIG. 2B, transversely connected vugs exhibit connectedness in first dimension 208 and second dimension 210 (e.g., vugs are connected to one another in at least two dimensions). In FIG. 2C, omnidirectional vugs exhibit connectedness in at least three dimensions 212, 214, 216.

Modelling engine 120 may be configured to utilize different vug types to model aspects of the permeability of a rock formation, as depicted in FIGS. 2A-2C. For example, as shown in FIGS. 2A-2C, a total reservoir volume may be determined by dividing the volume into smaller dimensions, such as cubes (as depicted in each of FIGS. 2A-2C), and a combination of many such smaller dimension volumes may be used to estimate the permeability of the total reservoir volume. In an aspect, the cubes shown in FIGS. 2A-2C may represent a volume of one cubic meter. However, the modelling engine 120 may be configured to utilize other dimensions, such as 0.5 meters, 2 meters, 10 meters, etc., depending on the particular configuration of modelling engine 120 and system 100. Modelling engine 120 may determine a largest connected volume (LCV) of vugs present within the smaller volume of the reservoir. The LCV corresponds to a value of a volume of the largest connected network of vugs in a volume. Additionally, modelling engine 120 may model the connectivity of vugs (e.g., vug connectivity) as LCV %, a continuous variable corresponding to a ratio between LCV volume and a total volume of a modelled reservoir. In an implementation, modelling engine 120 may model vugs as having a particular shape. For instance, modelling engine 120 may model vugs as having an oblate ellipsoid shape, as having a prolate ellipsoid shape, as having a spherical shape, and/or as having other shapes.

Modelling engine 120 may further be configured to derive vug attribute parameters from input data 190. Vug attribute parameters may include vug quantity (e.g., vug abundance), vug size, and/or vug aspect ratio. In implementations, vug quantity may correspond to a mean quantity of vugs present in a rock formation, vug size may correspond to a mean size of vugs present in the rock formation, and vug aspect ratio may correspond to a ratio of a first equatorial dimension of a vug shape to a second polar dimension of the vug shape of vugs present in the rock formation. In some aspects, the vug attribute parameters may include multiple values representing different portions of a formation under analysis. To illustrate, multiple vug quantities, vug sizes, vug aspect ratios (or alternatively shapes, which may be predicted based on the vug aspect ratio(s)), or other types of parameters may be specified for use by modelling engine 120, which may enable a more accurate understanding of the rock formation undergoing analysis.

In an implementation, modelling engine 120 may derive vug shape from input data 190, from vug parameters, or both. For example, based on input data 190 and/or derived vug parameters, modelling engine 120 may model vugs of a rock formation as having an oblate ellipsoid shape, as having a prolate ellipsoid shape, or as having a spherical shape. Additionally or alternatively, modelling engine 120 may receive vug shape as an input from a user of vug analysis device 110. For instance, a user of vug analysis device 110 may indicate, using I/O device 124, that vugs of a rock formation have an oblate ellipsoid shape, a prolate ellipsoid shape, or a spherical shape. Additionally or alternatively, although vug aspect ratio may be derived from input data 190, in implementations, a user of vug analysis device 110 may provide aspect ratio information as input to modelling engine 110. For instance, a user of vug analysis device 110 may input (using an I/O device 124) values corresponding to a first dimension (e.g., an equatorial dimension) and to the second dimension (e.g., a polar dimension) of a characteristic vug shape.

Modelling engine 120 may further be configured to generate modelled rock property data 126 based on executing modelling engine 120 against one or more of the vug attribute parameters. Modelled rock property data 126 may include a prediction of a permeability of the material under investigation (e.g., a prediction of the permeability of rock associated with a reservoir), such as a probability that the material under investigation (e.g., rocks of a rock formation) is permeable. For example, modelled rock property data 126 may include a probability that vugs of a rock formation are connected (e.g., that vugs of an LCV of touching vugs form a transverse vug passageway or an omnidirectional vug passageway). Additionally or alternatively, modelled rock property data 126 may include a probability that vugs of the rock formation are isolated.

Modelling engine 120 may generate vug connectedness predictions (e.g., vug connectedness probabilities) by performing a statistical analysis based, at least in part, on the vug parameters. For example, modelling engine 120 may be configured to perform binary logistic regression. In other examples, modelling engine 120 may be configured to perform log-binomial regression, Poisson regression, Poisson regression with a robust variance estimator, and/or Cox regression. Additionally or alternatively, modelling engine 120 may be configured to perform tree-based data analysis techniques, neural network-based analysis techniques including those implemented with support vector machines and/or a k-nearest neighbor algorithm. In this manner, modelling engine 120 may be configured to generate vug connectedness predictions (e.g., vug connectedness probabilities) based on a statistical analysis of vug parameters.

As a particular example, the regression may be binary logistic regression. Modelling engine 120 may be configured to evaluate the binary logistic regression, which may be expressed as:

P=Exp(C+[^{Ab}β*Ab]+[^{Ar}β*Ar]+[^{Si}β*Si]) (Equation 1)

where P represents the probability of LCVs serving as vug passageways; Exp represents antilogarithm (the inverse function of a logarithm); C is the regression constant; Ab, Ar, Si represent independent variables—abundance, aspect ratio, and size of vugs, respectively; and ^{Ab}β, ^{Ar}β, ^{si}β represent regression coefficients of abundance, aspect ratio, and size of vugs, respectively. Equation 1 above may be used to determine the probability (P) that an LCV comprises a vug passageway (e.g., transverse vug passageway or an omnidirectional vug passageway).

The logistic regression shown in Equation 1, with computed coefficients, can be used to calculate the probability that vugs form connected passageways. To make the prediction, size, shape and abundance of vugs must be known. Including the variables in the general form (of Equation 1 above) with the outcome from the logistic regression model, the probability of LCVs representing vug passageways for oblate vugs (Equation 2) or prolate vugs (Equation 3) can be calculated as follows:

P=Exp(−24.139)+[0.702*Ab]+[2.857*Ar]+[0.425*Si]) (Equation 2)

P=Exp(−28.803)+[0.973*Ab]+[3.147*Ar]+[0.081*Si]) (Equation 3)

Modelling engine 120 may further be configured to discretize the vug connectedness probabilities. For instance, modelling engine 120 may generate the discretized vug connectedness probability from a continuous vug connectedness probability by applying a threshold to the continuous vug connectedness probability such that any probability value below the threshold constitutes isolated vugs while any probability above the threshold constitutes a vug passageway (e.g., a transverse vug passageway, an omnidirectional vug passageway, etc.). In an example, a user of vug analysis device 110 may set the threshold, which may be stored in database 118. Thus, modelled rock property data 126 may include continuous vug connectedness probabilities in addition to discretized vug connectedness probabilities.

Vug analysis device 110 may be configured to display modelled rock property data 126 on I/O device 124 (e.g., a monitor). Additionally or alternatively, vug analysis device 110 may be configured to send modelled rock property data 126 to other devices, such as to at least one user device 134 of a plurality of user devices. For instance, vug analysis device 110 may be configured to send modelled rock property data 126 to other devices (e.g., to user device 130) via network 150. Alternatively or additionally, vug analysis device 110 may be configured to send modelled rock property data 126 directly to user device 130.

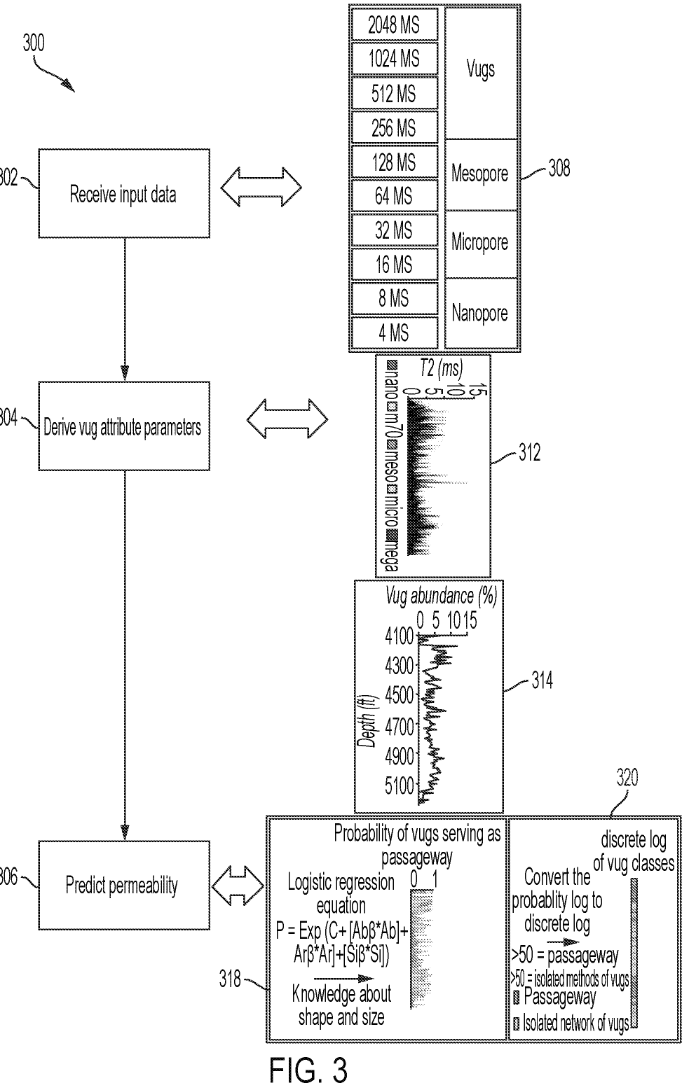

Referring briefly to FIG. 3, a flow diagram illustrating aspects of a permeability prediction process is shown. Vug analysis device 110 may be configured to execute the process depicted in FIG. 3. For instance, input data 190, such as NMR logs 308 may be received at communication interface 122 of vug analysis device 110. NMR logs 308 may be stored at and/or generated at a data source or a plurality of data sources such as data source 160, 170, and/or 180 and provided to vug analysis device 110 via network 150. NMR logs 308 may indicate the presence of vugs in rocks of a reservoir under investigation. Processor 112 may cause the NMR logs 308 to be stored in database 118 of memory 114 for additional and subsequent processing. Modelling engine 120 may be configured to derive vug attribute parameters, such as pore size distribution 312 of vugs in the rock formation, based on NMR logs 308. Modelling engine 120 may be further configured to derive vug abundance 314 from the pore size distribution 312 (e.g., expressed as a percentage of a volume of a rock formation that include vugs as a function of a depth of the rocks of the rock formation). Additionally, modelling engine 120 may be configured to perform a statistical analysis of vug abundance 314 to generate vug connectedness probability 318. Vug connectedness probability 318 may indicate a probability that vugs at a particular depth within rocks of a rock formation constitute vug passageways (e.g., are transverse vugs and/or omnidirectional vugs). Modelling engine 120 may be further configured to convert vug connectedness probability 318 to discrete vug connectedness probability 320 by applying a threshold such that any probabilities below the threshold are interpreted as isolated vugs, while any probabilities above the threshold are interpreted as vug passageways (e.g., constituting transverse and/or omnidirectional vugs).

Vug connectedness probability 318 and discrete vug connectedness probability may comprise modelled rock property data 126. Discrete vug connectedness probability 320 may be used to identify depths at which rock formations are likely to contain vug passageways or depths of rock formations that are likely to contain isolated vugs. In some applications, the likely presence, in rock formations, of vug passageways may be indicative of a location at which to drill and extract or inject fluids (e.g., to extract fluids such as hydrocarbons and/or water present in the rock formation and flowing within permeable portions of a reservoir or inject waste product such as carbon dioxide or contaminated water). In other applications, the likely presence, in rock formations, of isolated vugs may be indicative of locations, within a rock formation, at which to assess stability of injected waste materials (e.g., carbon dioxide, nuclear waste, etc.) to reduce a likelihood that the sequestered waste will rise to the surface or into another rock unit.

Referring back to FIG. 1, although material permeability prediction system 100 is depicted as having a vug analysis device 110, in implementations, material permeability prediction system 100 may include a network of vug analysis devices, such as vug analysis device 110. The network of vug analysis devices may be configured to process input data 190 in parallel and to combine the results of the processing to conserve computational resources. In this manner, the network of vug analysis devices may predict the permeability of a material (e.g., a rock formation or a reservoir) in less time than would be expended by a single vug analysis device 110 to generate the prediction.

To illustrate, a network of vug analysis devices may include a first vug analysis device and a second vug analysis device. The first vug analysis device and the second vug analysis device may each be configured to receive input data 190. The first vug analysis device and the second vug analysis device may each be configured to derive vug attribute parameters from input data 190. The first vug analysis device may perform a first set of processing steps on the vug attribute parameters. The second vug analysis device may, in parallel to the first vug analysis device, perform a second set of processing steps, distinct from the first set of processing steps, on the vug attribute parameters. The first vug analysis device may send the results of the first set of processing steps to the second vug analysis device. The second vug analysis device may combine the results of the first set of processing steps with the results of the second set of processing steps to generate the prediction (e.g., a probability associated with vug connectedness). In this manner, parallel processing of input data 190 may reduce processing time and lead to a more rapid permeability prediction.

As briefly described above, FIGS. 2A-2C are images illustrating vug network models that may be generated in accordance with aspects of the present disclosure, shown as vug network models 202, 204, and/or 206, respectively. Vug network models 202, 204, and/or 206 may be models representing an LCV of vugs with a defined volume of a rock formation or material. A modelling engine (e.g., modelling engine 120 of FIG. 1) of a vug analysis device (e.g., vug analysis device 110) may be configured to generate vug network models 202, 204, and/or 206. In an implementation, vug network models 202, 204, and/or 206 may model the connectivity of vugs (e.g., vug connectivity) as LCV %, a continuous variable corresponding to a ratio between LCV volume and a total volume of a modelled reservoir containing vugs.

As an example, the modelling engine (e.g., modelling engine 120 of FIG. 1) may generate one of vug network models 202, 204, or 206 based on vug parameters (e.g., vug quantity, vug size, and/or vug aspect ratio). The modelling engine may derive the vug parameters from input data, such as NMR logs, CT images, etc. associated with rocks of the rock formation being modelled. The modelling engine may model each vug as having a particular shape (e.g., oblate ellipsoid, prolate ellipsoid, sphere, etc.).

As an example, FIG. 2A illustrates vug network model 202 simulating isolated vugs of a simulated reservoir. The vugs illustrated in FIG. 2A may be understood to represent an isolated LCV of vugs due to the fact that the vugs present in the volume modelled in FIG. 2A are embedded entirely with the modelled volume and are not connected to any of the portions of the volume surrounding the modelled portion of the volume. FIG. 2B depicts vug network model 204 simulating transverse vug passageways of a simulated reservoir. As shown in FIG. 2B, the simulated vugs are connected in two different dimensions, such as first dimension 208 and second dimension 210. Thus, the transverse vug passageway shown in FIG. 2B represents a portion of a volume of a formation or material that is permeable with respect material or portions of the volume adjacent to the modelled portion of the volume in two dimensions. It is noted that a transverse vug passageway may be connected to adjacent volumes in different dimensions than illustrated in FIG. 2B, such as the top and bottom, top and a side, bottom and a side, or different sides than first and second dimensions 208, 210. In FIG. 2C, a vug network model 206 simulating omnidirectional vug passageways of a simulated vug reservoir. The omnidirectional vug passageways are connected in three or more dimensions (sides of the model), such as first dimension 212, second dimension 214, and third dimension 216.

Referring to FIG. 3, a flow diagram illustrating aspects of a permeability prediction process is shown as method 300. At block 302 input data is received. In an implementation, the input data may be input data 190 of FIG. 1 and may be received at a vug analysis device, such as vug analysis device 110 of FIG. 1. The input data may be generated at and received from a plurality of data sources, such as data sources 160, 170, and 180 of FIG. 1. In an example, a data source may be a sensor positioned in a downhole well of a rock formation. The sensor may include instruments to collect data characterizing the rocks of the rock formation. For instance, the sensor may include an NMR device and may generate NMR log 308 characteristic of rocks of a rock formation. The sensor may include different or additional instruments capable of capturing other data about the rocks of the rock formation. For instance, the sensor may include a sonic image logging device to collect a sonogram of the rocks of the rock formation, a CT scanner to collect CT data characteristic of rocks of the rock formation, electrical equipment to collect electrical characteristics (e.g., impedances) of rocks of the rock formation, and/or optical equipment (e.g., infrared, ultraviolet, and/or visible light spectrometers) to collect other optical data characteristic of the rocks of the rock formation.

As another example, a data source may be a computer memory storing data about rocks of the rock formation, such as NMR logs, image logs, and/or other data characteristic of rocks of the rock formation. Additionally or alternatively, the data source may be an autonomous vehicle (e.g., a drone, rover, etc.) having instruments capable of gathering data about rocks of the rock formation. For instance, the autonomous vehicle may be configured with ground penetrating radar capable of generating image data about rocks of the rock formation.

At block 304, vug attribute parameters may be derived from the input data. For example, a vug analysis device, such as vug analysis device 110 of FIG. 1, may derive, using processor 112 of FIG. 1 executing vug modelling engine 120, vug attribute parameters based on the input data. Vug attribute parameters may include pore size distribution 312 of rocks of a rock formation. T2 relaxation time of the NMR provides a proxy for pore size. Based on the T2 bins, such as those corresponding to the bins of NMR logs 308 of FIG. 3, different classes of pores can be assigned (e.g., micro, meso, and vugs). By knowing the percentage of vugs from the pore size distribution, one can calculate vug abundance from total porosity. Other vug parameters may include a mean vug size (e.g., diameter) of vugs present in a rock formation, an aspect ratio of a vug shape (e.g., a ratio of a first equatorial diameter of the vug shape to a polar diameter of the vug shape), and/or a quantity of vugs present in rocks of a rock formation (e.g., expressed as a percentage of the rock formation constituting of vugs).

In an implementation, vug attribute parameters may be further derived from a particular vug attribute. For instance, vug abundance 314 may be derived based on pore size distribution 312. Vug abundance 314 may be expressed as a percentage of rocks of a rock formation that include vugs as a function of a depth of the rocks of the rock formation.

At block 306, permeability of a material under investigation is predicted based on a prediction of vug connectedness present in the material (e.g., rocks of a rock formation). For example, permeability of rocks of a rock formation is predicted based, at least in part, on determination of vug connectedness probability generated based on vug attribute parameters. In an implementation, a vug analysis device, such as vug analysis device 110 of FIG. 1, may be configured to predict material permeability based on vug connectedness probabilities determined from vug attribute parameters. In such an implementation, a modelling engine (e.g., modelling engine 120 of FIG. 1) of the vug analysis device may analyze the vug attribute parameters to generate vug connectedness probabilities indicative of a permeability of the vuggy material. For instance, the modelling engine may be configured to apply a statistical analysis (e.g., binary logistic regression) to generate vug connectedness probability 318.

Vug connectedness probability 318 comprises a probability that rocks of a rock formation include a vug passageway (e.g., a transverse passageway and/or an omnidirectional vug passageway). In other implementations vug connectedness probability 318 may comprise a probability that rocks of a rock formation contain isolated vugs (e.g., as opposed to connected vug passageways). Additionally, the modelling engine may be configured to discretize vug connectedness probability 318. For instance, the modelling engine may be configured to convert vug connectedness probability 318 to discrete vug connectedness probability 320 by applying a threshold such that if a probability is below the threshold, then the vugs correspond to isolated vugs, while if a probability is above the threshold, then the vugs correspond to vug passageways (e.g., a transverse passageway and/or an omnidirectional vug passageway). The threshold may be set by a user of the vug analysis device, and the user of the vug analysis device may vary the threshold. Vug connectedness probability 318 and/or discrete vug connectedness probability 320 may comprise modelled rock property data (e.g., modelled rock property data 126 of FIG. 1).

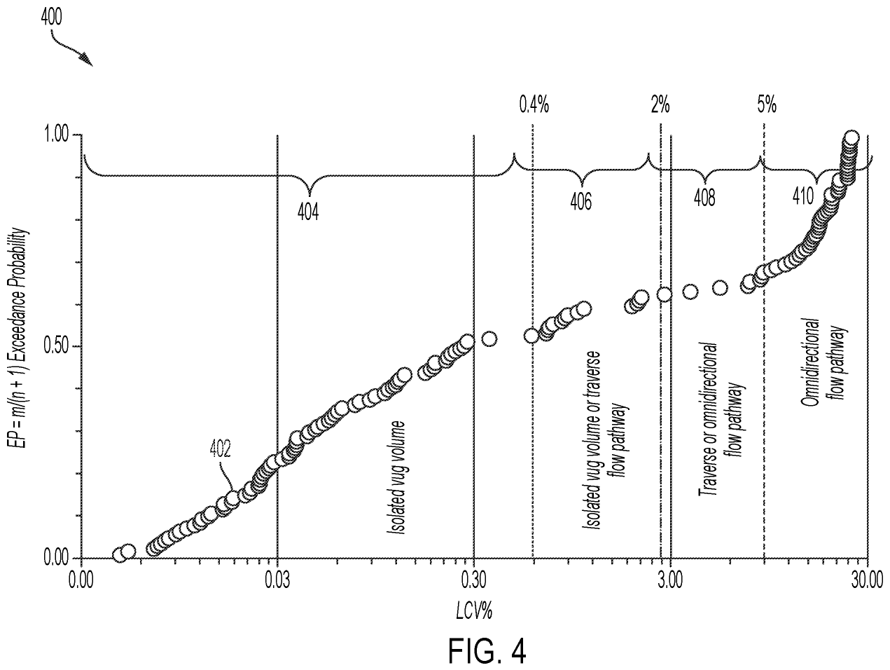

FIG. 4 depicts an example of probabilities of achieving different classes of permeability from networks of vugs with different attributes according to embodiments of the present disclosure. In the example of FIG. 4, modelled rock property data 400 includes a plot of exceedance probability against LCV % based on execution of a modelling engine against a plurality of vug parameters for each model instantiation 402. Exceedance probability characterizes a cumulative probability (e.g., P[X≥x])) of a certain LCV (e.g., LCV (X)) having an LCV % larger than a particular value (e.g., x). Thus, the y axis of FIG. 4 defines exceedance probability to be a rank, m, of a vug connectedness probability generated by each model instantiation 402 divided by a total number of model instantiations, n, executed by a modelling engine.

Accordingly, exceedance probability may be used as a predictor of a permeability of a material under investigation (e.g., rocks of a rock formation). As shown in FIG. 4, in first zone 404, exceedance probabilities less than or equal to 0.5 correspond to LCV % less than or equal to 0.4% and are indicative of LCVs that correspond to isolated vugs. In second zone 406, exceedance probabilities greater than 0.5 and less than 0.6 correspond to LCV % values greater than 0.4% and less than 2% and are indicative of LCVs that correspond to isolated vugs or transverse vug passageways. In third zone 408, exceedance probabilities greater than 0.5 and less than 0.6 correspond to LCV % values greater than 2% and less than 6% and are indicative of LCVs that correspond to transverse vug passageways or omnidirectional vug passageways. In fourth zone 410, exceedance probabilities greater than correspond to LCV % values greater than 5% and are indicative of LCVs that correspond to omnidirectional vug passageways.

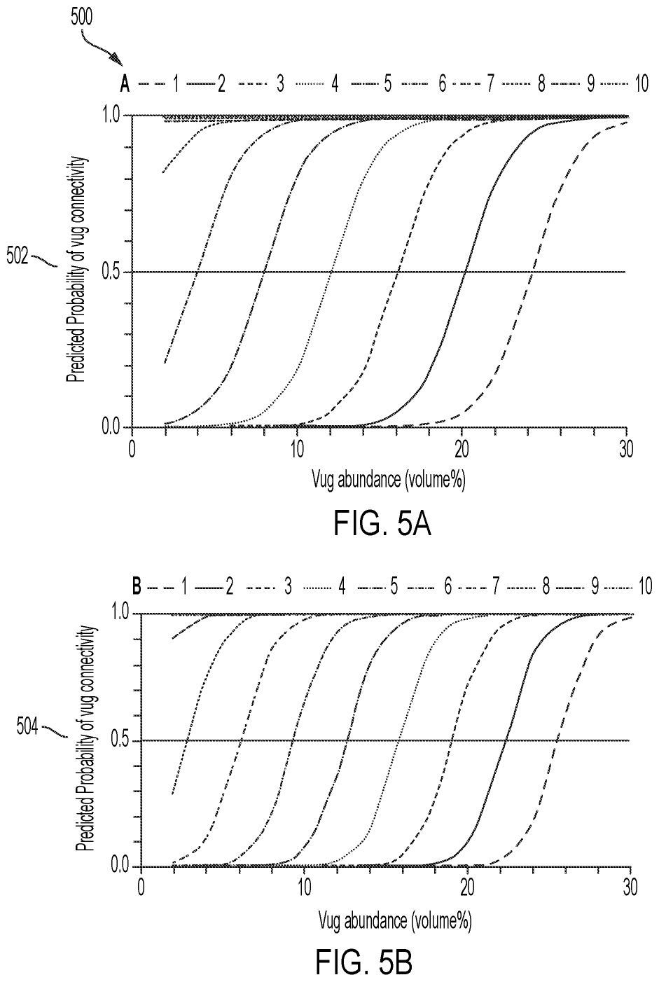

FIGS. 5A and 5B depict additional examples of probabilities of achieving permeability from networks of vugs given different vug attributes according to embodiments of the present disclosure. In the examples of FIGS. 5A and 5B, a modelling engine (e.g., modelling engine 120 of FIG. 1) is configured to generate modelled rock property data 500 by maintaining vug size as a constant (e.g., with vugs modelled as having a diameter of ten units in a coordinate system of the model). In FIG. 5A, the modelling engine (e.g., modelling engine 120 of FIG. 1) may be configured to model a vug as having an oblate ellipsoid shape. By contrast, in FIG. 5B, the modelling engine (e.g., modelling engine 120 of FIG. 1) may be configured to model a vug as having a prolate ellipsoid shape.

In generating nomograms 502 and 504, an aspect ratio of a representative vug shape (e.g., the oblate ellipsoid of FIG. 5A, the prolate ellipsoid of FIG. 5B) may be varied to produce the plurality of curves depicted in FIGS. 5A and 5B. For instance, a modelling engine executing a model to generate nomograms 502, 504 may derive, as vug attribute parameters, a first value corresponding to an equatorial diameter of the representative vug shape (e.g., the oblate ellipsoid of FIG. 5A, the prolate ellipsoid of FIG. 5B) and a second value corresponding to a polar diameter of the representative vug shape (e.g., the oblate ellipsoid of FIG. 5A, the prolate ellipsoid of FIG. 5B) from input data. In other implementations, a user of a vug analysis device (e.g., vug analysis device 110), executing the model, may provide the first value and the second value as inputs to the modelling engine.

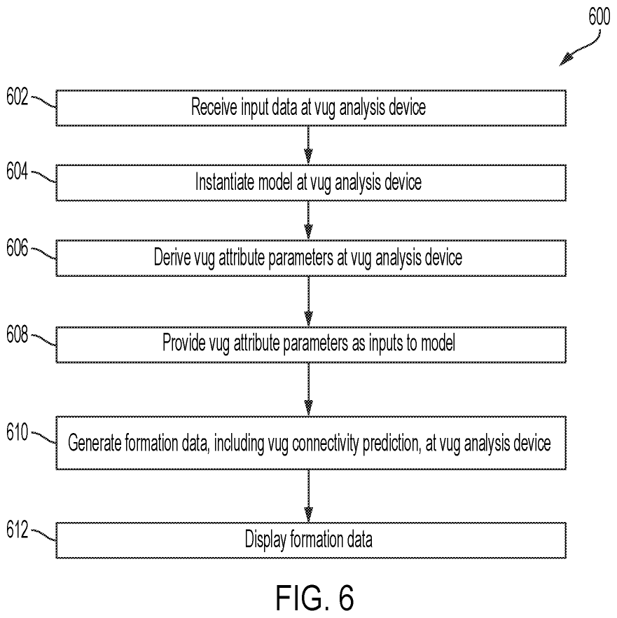

Referring to FIG. 6, a process for modelling permeability from vug attributes according to embodiments of the present disclosure is shown as a method 600. At block 602, the method 600 includes receiving input data at a vug analysis device, such as vug analysis device 110 of FIG. 1. For example, input data may be received at communication interface 122 of vug analysis device 110 as shown in FIG. 1. Input data may be received at the vug analysis device from data sources of a plurality of data sources. A first data source may include a database on a computing device that stores physical data corresponding to a material under investigation, such as a database storing physical data corresponding to rocks of a rock formation. A second data source may be a sensor disposed within a material under investigation (e.g., rocks of a rock formation) equipped with instruments to gather physical and/or chemical data about the material under investigation. A third data source may be LiDAR, photogrammetry, autonomous vehicle, or the like equipped with instruments to gather data about the material under investigation, such as rocks of a rock formation. The data sources are not limited to three data sources. Any number of a plurality of data sources may provide input data to the vug analysis device.

Input data may comprise physical and/or chemical data about the material under investigation, such as physical and/or chemical data associated with rocks of a rock formation. The physical and/or chemical data may include NMR logs, image logs, and/or other compilations of physical and/or chemical data about the material under investigation. Additionally, input data may include electrical data about the material under investigation, such as impedance data; seismic data; and optical data, such as visible and non-visible light spectroscopic analysis results.

At block 604, the method 600 includes a model instantiated at the vug analysis device. For example, modelling engine 120 of vug analysis device 110 of FIG. 1 may be configured to generate a model of rocks, rock formations, and/or reservoirs. To illustrate and as an example, the modelling engine of the vug analysis device may generate a simulation of rocks, rock formation, and a reservoir, such as the simulations shown in FIG. 2. The simulation may simulate geophysical characteristics of the rocks, the rock formation, and/or the reservoir.

At block 606, the method 600 includes vug attribute parameters derived at the vug analysis device. For example, processor 112 and/or modelling engine 120 of vug analysis device 110, as depicted in FIG. 1, may be configured to derive vug attribute parameters based, at least in part, on input data. Vug attribute parameters may include a representative vug shape, a representative vug size (e.g., a vug diameter), a representative vug aspect ratio (e.g., a ratio of a first dimension of the representative vug shape to a second dimension of the representative vug shape), and/or a quantity of vugs present in a rock formation (e.g., a vug abundance). To illustrate, input data may include NMR logs comprising NMR spectra of rocks of a rock formation. A processor and/or modelling engine of a vug analysis device may be configured to derive a distribution of vug diameters of vugs present in the rocks of the rock formation from the NMR logs. The processor and/or modelling engine may be further configured to derive a vug abundance based on the distribution of vug diameters. The vug abundance may be expressed as a percentage of vugs comprising a total volume of the rock formation and/or reservoir.

At block 608, the method 600 includes vug attribute parameters provided as inputs to the model. In an example, modelling engine 120 of vug analysis device 110, as depicted in FIG. 1, may receive vug attribute parameters. Modelling engine 120 may be configured to statistically analyze vugs present in rocks of a rock formation based, at least in part, on the vug attribute parameters. For instance, modelling engine 120 may be configured to provide vug attribute parameters as inputs to a statistical model of vug behavior. As a particular example, the statistical model may be any of a binary logistic regression algorithm, a polytomous regression algorithm, a conditional logistic regression algorithm, and/or any other statistical algorithm.

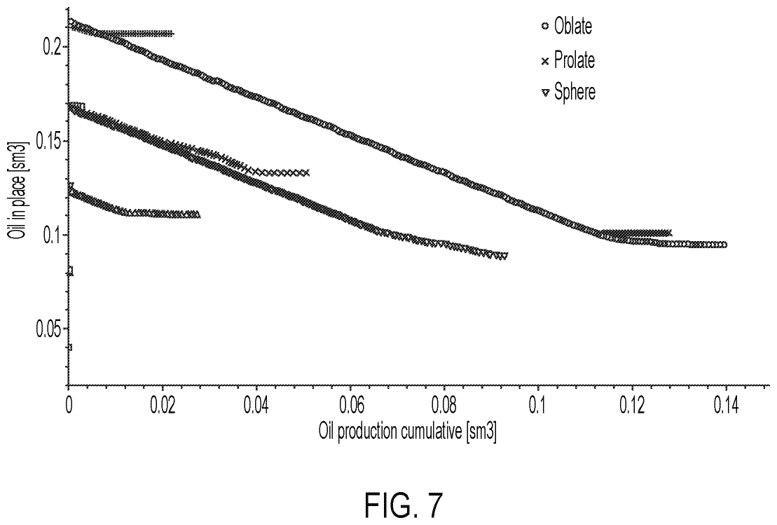

At block 610, the method 600 includes modelled rock property data, including a vug connectivity prediction, is generated at a vug analysis device, such as vug analysis device 110 of FIG. 1. As an example, an output of the statistical model may be a probability that vugs of a rock formation comprise vug passageways. Alternatively, an output of the statistical model may be a probability that vugs of the rock formation comprise isolated vugs. In an implementation, the probability of vug connectedness may be discretized by applying a threshold such that any probability below the threshold is interpreted as meaning that vugs of a rock formation comprise isolated vugs, while any probability above the threshold is interpreted as meaning that vugs of the rock formation comprise a vug passageway. Additionally, modelled rock property data may include outputs such as those described in FIGS. 4, 5A, 5B, 7, 9A, 9B, 10A, and/or 10B. In a particular implementation, the vug analysis device (e.g., vug analysis device 110) may be configured to determine, based on the probability of vug connectedness, a type of fluid flow within a reservoir as described in the context of FIG. 7.

At block 612, the method 600 includes displaying the modelled rock property data. In an example, modelled rock property data may be displayed at a display device, such as a display device of the one or more I/O devices 124 of vug analysis device 110 of FIG. 1 or a display device of the I/O devices 138 of user device 130 of FIG. 1.

Method 600 has numerous advantages. First, method 600 accounts for micro and macro properties of vugs, thereby generating more accurate predictions of vug connectedness and, by extension, material permeability, than traditional methods. Second, method 600 accounts for vug geometry (e.g., vug shape) in assessing vug connectedness and, by extension, material permeability. By accounting for vug geometry (e.g., vug shape and aspect ratio), method 600 generates more accurate predictions of vug connectedness (and by extension material permeability) than traditional methods. Third, method 600 enables a prediction of a type of fluid flow in a reservoir thereby highly correlating with a cumulative production of an oil and gas reservoir, a suitability of a reservoir for the sequestration of carbon dioxide, a flow rate of water through a reservoir, etc. Since method 600 accurately predicts vug connectivity and, by extension, material permeability (e.g., permeability of rocks of a rock reservoir), method 600 may be deployed to identify productive hydrocarbon reservoirs, water reservoirs, and/or reservoirs that may be suitable for sequestration or storage of gases, liquids, and other materials (e.g., wastes such as carbon dioxide, contaminated water, etc., or storage of natural gas, compressed air, or hydrogen). Fourth, method 600 could be easily integrated into existing workflows (e.g., by using commonly available well logging data) to generate vug connectedness predictions (and, by extension, material permeability predictions). The foregoing enumeration is not intended to be limiting, and it is understood the method 600 includes other advantages not explicitly delineated herein.

FIG. 7 depicts that predictions of material permeability based on vug connectedness probabilities correlate with a fluid flow rate of a reservoir. For instance, as shown in FIG. 7, the modelling engine (e.g., modelling engine 120 of FIG. 1) may be configured to model vugs of a vug reservoir as having an oblate ellipsoid shape and having any one of 5% vug abundance, 10% vug abundance, 15% vug abundance, 20% vug abundance, and 25% vug abundance as a percentage of a total volume of the simulated reservoir. In a second set of vug modelling results, the modelling engine may be configured to model vugs of a vug reservoir as having a prolate ellipsoid shape with the same percentages of vug abundance as in the first set of modelling results. In a third set of vug modelling results, the modelling engine may be configured to model vugs of a vuggy reservoir as having a spherical shape with the same percentages of vug abundance as in the first and second sets of modelling results.

As depicted in FIG. 7, reservoirs having an abundance of oblate ellipsoid or prolate ellipsoid vugs (thereby exhibiting a high probability of vug connectedness and thus exhibiting a high probability of rock permeability) show a high degree of correlation with hydrocarbon (e.g., oil) production. In this manner, FIG. 7 shows that method 600 may be used to identify reservoirs having permeable rock formations.

In a particular implementation, the disclosure includes an apparatus for predicting material permeability, such as permeability of rocks of a rock formation. The apparatus may include means for receiving input data. The means for receiving input data may correspond to communication interface 122 of FIG. 1. The input data may include data received from one or more data sources, such as data sources 160, 170, and 180 of FIG. 1.

The apparatus may further include means for storing instructions and the input data. The means for storing may correspond to memory 114 of FIG. 1, and the input data may be stored in a database 118 of the means for storing. The instructions may comprise computer-readable instructions corresponding to instructions 116 of FIG. 1.

Additionally, the disclosure may include a means for processing. The means for processing may correspond to processor 112 of FIG. 1. The means for processing may be configured to execute computer-instructions stored in the memory to derive vug attribute parameters from the input data and to generate a model of vugs comprising a rock formation based, at least in part, on the vug attribute parameters. Moreover, the means for processing may be further configured to generate modelled rock property data that includes a probability indicative of vug connectedness based, at least in part, on the model. Moreover, the apparatus may include means for displaying. The means for display may correspond to I/O device 124 of FIG. 1. The means for displaying may be configured to display the modelled rock property data.

### Experimental Results

The following paragraphs describe experimental results to provide a more complete understanding of the disclosed devices, systems and methods. It is appreciated that these experimental results are provided for purposes of illustrating the accuracy and other aspects of the concepts disclosed herein, rather than by way of limitation. Furthermore, it is to be appreciated that the exemplary experiments described below illustrate specific examples of how the permeability prediction techniques disclosed herein may be applied, but that embodiments of the present disclosure may be utilized in other types of reservoirs, rock formations, or situations and applications where pore, particle, or object connectedness must be determined, and purposes that may differ from the exemplary experimental results described below.

### Experiment 1

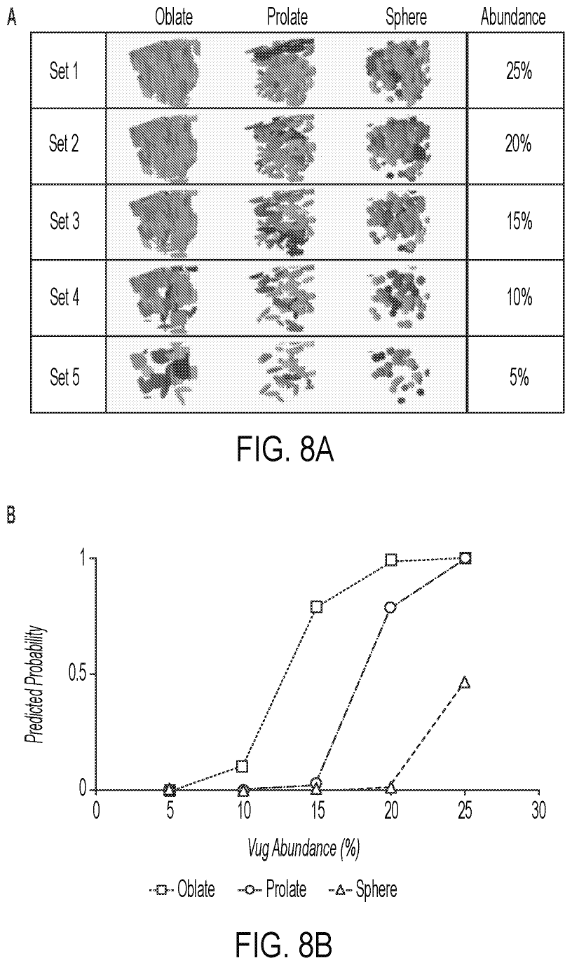

Referring to FIG. 8A, vugs were modelled on a computing device. Five different sets of models were executed. Each set was executed with the following vug attribute parameters: a representative vug shape corresponding to an oblate ellipsoid, a prolate ellipsoid and a sphere; an aspect ratio of three; and vug abundances ranging from 25% for set 1 to 5% for set 5 with vug abundance being diminished by 5% for each successive set starting with set 1. In executing the vug network models and referring to FIG. 8B, the computing device generated probabilities that the vugs comprised vug passageways (e.g., transverse vug passageways and/or omnidirectional vug passageways) as a function of vug abundance. As shown in FIG. 8B, a probability that vugs comprise vug passageways increases exponentially with increased vug abundance and assuming that each vug has either an oblate ellipsoid shape or a prolate ellipsoid shape.

### Experiment 2

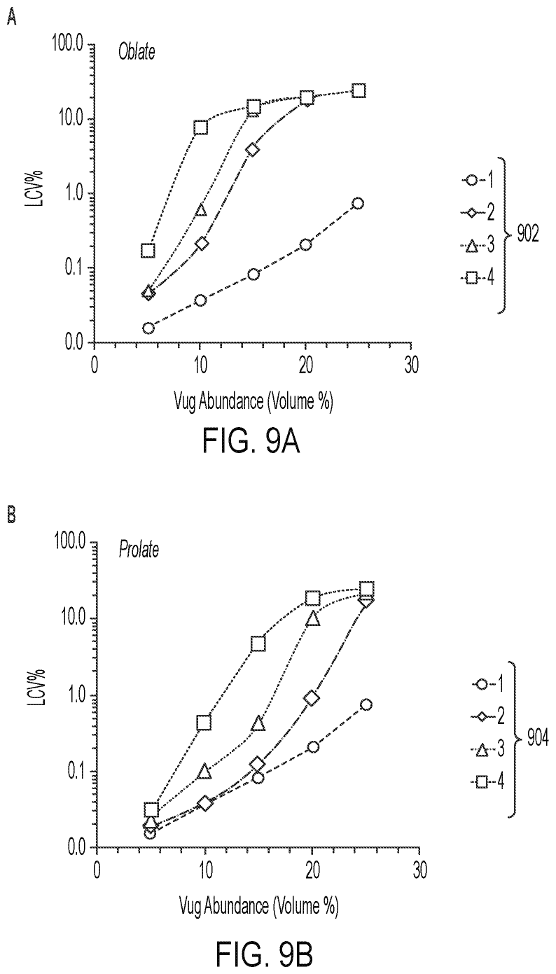

Referring to FIGS. 9A and 9B, a computing device modelled LCV % as a function of vug abundance (as a percentage of a total volume of a simulated reservoir) assuming four different aspect ratios. In FIG. 9A, the computing device was configured to model vug shape as an oblate ellipsoid with first four aspect ratios 902. One aspect ratio of first four aspect ratios 902 was one, which corresponds to a spherical shape (e.g., having equal equatorial and polar dimensions). In FIG. 9B, the computing device was configured to model vug shape as an elliptical ellipsoid with second four aspect ratios 904. As in FIG. 9A, one of the aspect ratios of second four aspect ratios was one, corresponding to spherically shaped vugs.

### Experiment 3

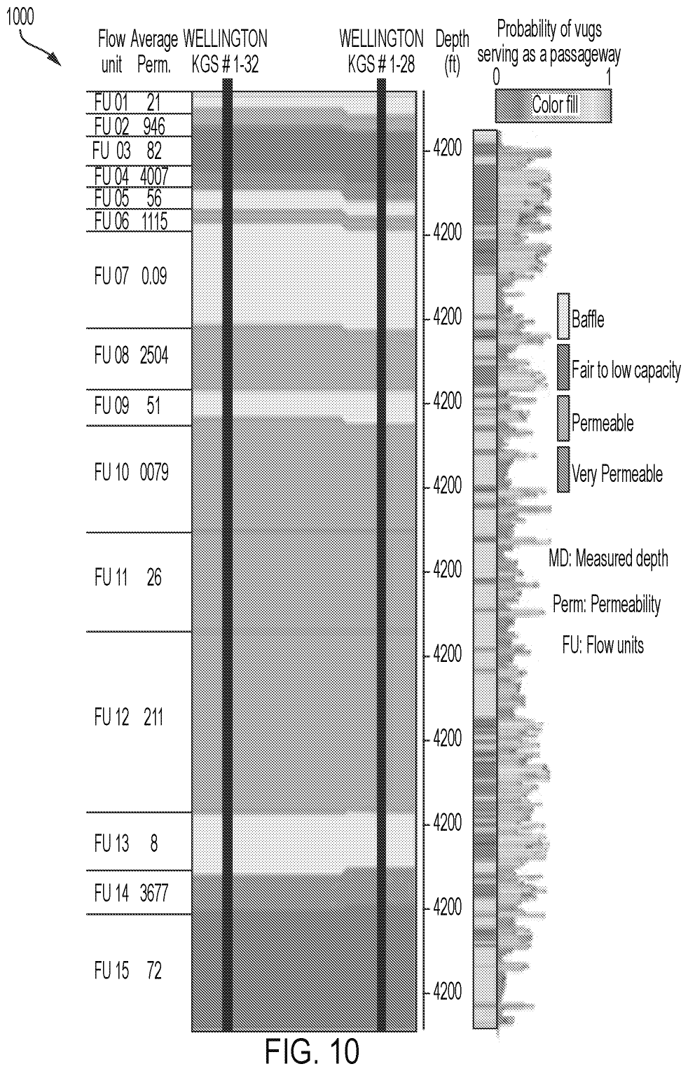

FIG. 10 depicts results of an experiment in which a computing device was used to model and predict vug connectivity in the Arbuckle Group at the Wellington Field in Kansas. NMR log data associated with the Wellington Field was provided as an input to the computing device. Based on the input data (e.g., the NMR log data), the computing device was configured to determine vug attribute parameters. Using binary logistic regression, the computing device calculated probabilities that vugs in the Wellington Field comprised vug passageways. The probabilities were compared against permeability data associated with the Wellington Field. Flow units of the Wellington Field demonstrating excellent permeability (e.g., permeability greater than 1 Darcy) were predicted by the model to have vug passageways. All flow units of the Wellington Field showing poor permeability (e.g., less than 20 millidarcy (mD)) were predicted by the model to comprise isolated vugs. Accordingly, experimental permeability data was used to validate the model and demonstrate the ability of models according to embodiments to accurately predict permeability of formations based on the presence of vugs and their connectedness.

### Experiment 4

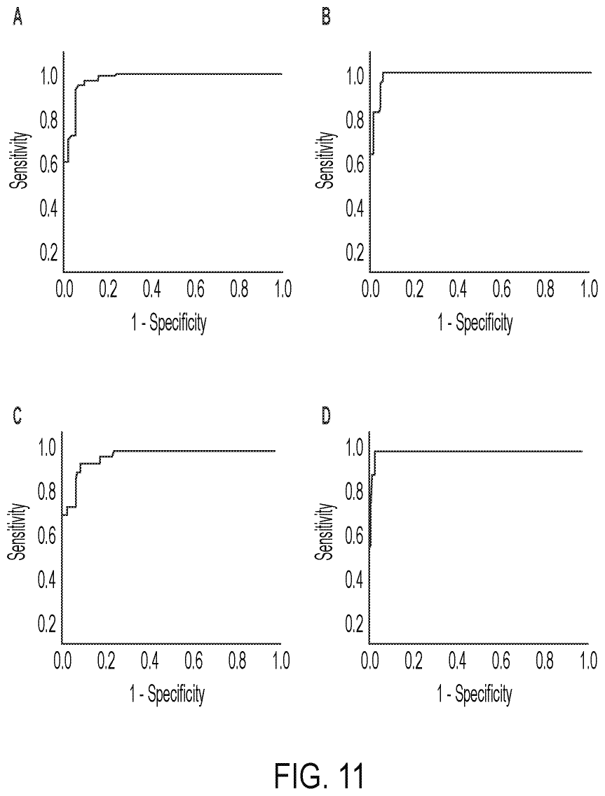

FIG. 11 depicts receiver operating characteristic (ROC) curves prepared for simulations of vuggy rock formations in which a computing device used binary logistic regression to determine a probability that vugs connected to form a vug passageway. In the ROC curves, sensitivity is defined as an ability of the model to correctly identify vugs as comprising a vug passageway. Specificity is defined as the ability of the model to correctly identify that vugs are isolated. The results showed that a sensitivity of the model was 96.5% when the model simulated vugs as having an oblate ellipsoid shape and 100% when the model simulated vugs as having a prolate ellipsoid shape. The specificity of the model was 95% when the model simulated the vug as having an oblate ellipsoid shape or a prolate ellipsoid shape. The area under the ROC curve for observed versus predicted vug passageways was 0.98 when the model simulated the vugs as having a prolate ellipsoid shape and 0.97 when the model simulated the vugs as having an oblate ellipsoid shape with a P-value<0.05 for both vug topologies.

Despite the importance, characterizing vugs, and their impact on flow, has proven challenging, and such efforts commonly do not match with production history. Many such prediction challenges relate to the inability of core plug and whole core analysis to capture the large scale of vug passageways, and therefore, the permeability created by (and flow through) networks of connected vugs. The small scale of typical measurements in subsurface examples may be the major shortcoming. A typical core with vugs that could be characterized by core plugs, full diameter (whole core) analysis, or computed tomography (CT) scans, typically includes vugs with a wide range of size, shape and distribution. This situation leads to core plugs that sample a volume too small to be representative, and typically only sample the small-scale vugs.

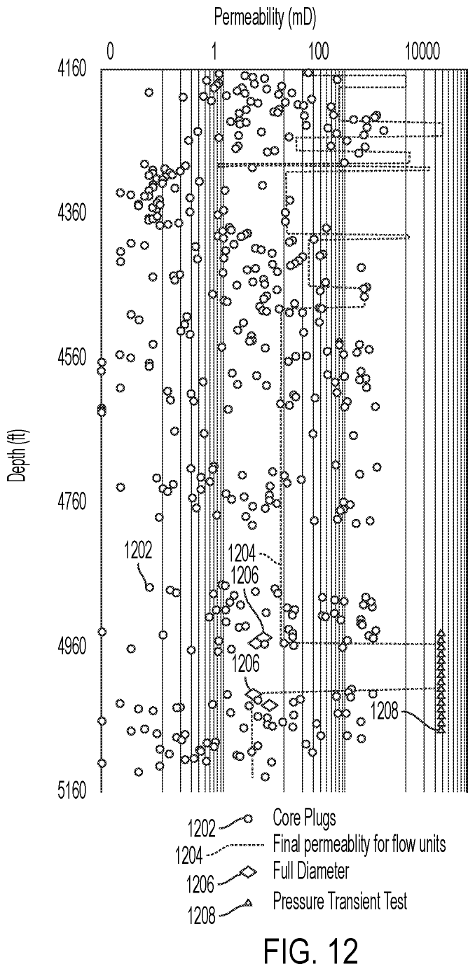

To illustrate and referring to FIG. 12, a diagram illustrating exemplary rock formation samples from the Arbuckle Group of Kansas is shown. In FIG. 12, core plugs 1202 of the vuggy intervals provided permeability values that were low, but highly variable. In contrast, full diameter core analyses 1206 of permeability offered higher or average values for the core segment, sampling more of the vugs. Nonetheless, pressure transient tests 1208 in the subsurface show that the actual formation permeabilities 1204 in the vuggy intervals are higher than either of the two types of measurements. In this reservoir, core plug and whole-core measurements in the tens of mD range compared poorly with pressure transient tests 1208 that indicate thousands of mD in the same stratigraphic interval. Considering that core CT scans and image logs all provide measurements at the core scale, it is clear that measurement of permeability at only core scale is not sufficient for characterizing this reservoir.

Understanding the pattern of vug connectivity using conceptualized numerical models provides insights into petrophysical assessment, reservoir characterization, and modeling of vuggy reservoirs. As described in more detail below, changes in vug attributes (e.g., vug shape, vug abundance and vug size) all drive changes in the probability that the vugs: (1) remain as isolated networks; (2) create networks of touching vugs that form transverse vug passageways; or (3) become networks of touching vugs with omnidirectional vug passageways. Each pattern likely has distinct petrophysical characteristics in terms of fluid flow behavior, and this behavior can be modeled using the logistic regression algorithms and techniques described above, thereby enabling more accurate measurements of permeability or other characteristics of a reservoir or rock formation.

To illustrate how the modelling techniques and concepts described provide a better understanding of performance of reservoirs, an analysis of variance (ANOVA) was performed to test the influence of abundance, shape, and size on LCV %. Results of the ANOVA are shown below in Table 1.

TABLE 1

| | | Type III | Degree | | | |
|---|---|---|---|---|---|---|
| | | Sum of | of | Mean | | |
| | Source | Squares | Freedom | Square | F-Test | p-value |
| Oblate | Intercept | 66284.9 | 79.0 | 839.0 | 4617.7 | 0.00 |
| | Vug Size | 40669.3 | 1.0 | 40669.3 | 223825.5 | 0.00 |
| | Vug Abundance | 621.5 | 3.0 | 207.2 | 1140.2 | 0.00 |
| | Aspect Ratio | 39413.0 | 4.0 | 9853.3 | 54227.8 | 0.00 |
| Prolate | Intercept | 47507.3 | 79.0 | 601.4 | 1642.5 | 0.00 |
| | Vug Size | 15708.9 | 1.0 | 15708.9 | 42906.5 | 0.00 |
| | Vug Abundance | 66.1 | 3.0 | 22.0 | 60.2 | 0.00 |
| | Aspect Ratio | 27125.0 | 4.0 | 6781.3 | 18522.0 | 0.00 |

The ANOVA results shown in Table 1 above indicate statistically significant relations for both oblate and prolate ellipsoids and point to a difference in the levels within each independent variable (e.g., p-value of all variables is <0.05). Note that each of these two shape classes include spheres as the 1:1:1 shape variant. As expected based on the graphical results (e.g., FIGS. 9A and 9B above) and ANOVA, binary logistic regression reveals that vug abundance, size, and shape are statistically significant predictors of LCV %.

These relations can be further assessed by use of the odds ratio in the results of binary logistic regression analysis. In binary logistic regression, odds ratio is a factor used to recognize the influence of a predictor on the dependent variable. In this analysis, the odds ratio is used as a proxy for the probability of the LCV to be an isolated network of vugs versus a vug passageway, the results of which are shown in Tables 2 and 3 for oblate and prolate, respectively.

TABLE 2

| | β-value | Standardized β-value | Odds ratio | p-value |
|---|---|---|---|---|
| Vug Size | 0.425 | 0.323 | 1.529 | 0.000 |
| Vug Abundance | 0.702 | 1.690 | 2.018 | 0.000 |
| Aspect Ratio | 2.857 | 1.085 | 17.408 | 0.000 |
| Constant | −24.139 | | 0.000 | 0.000 |

TABLE 3

| | β-value | Standardized β-value | Odds ratio | p-value |
|---|---|---|---|---|
| Vug Size | 0.081 | 0.064 | 1.084 | 0.298 |
| Vug Abundance | 0.973 | 2.453 | 2.645 | 0.000 |
| Aspect Ratio | 3.147 | 1.265 | 23.262 | 0.000 |
| Constant | −28.803 | | 0.000 | 0.000 |

This dataset revealed three scenarios based on the odds ratio for LCV serving as vug passageways: 1) An odds ratio=1 means the likelihood of having and not having a vug passageway in the model is equal; 2) An odds ratio>1 means that the likelihood of LCVs serving as vug passageways increases with increasing values of the independent variables; and 3) An odds ratio<1 means the likelihood of LCVs serving as vug passageways decreases with increasing values in the independent variables. The regression results for both shape families show that odds ratios of vug abundance, vug shape, and vug size are >1, indicating that the probability of having LCVs serve as vug passageways increases with increases in each of those variables—vug abundance, vug aspect ratio, and vug size.

In the binary logistic regressions described above, the standardized regression coefficient of the independent variables (i.e., standardized β value, Tables 2 and 3) define the weight, or how much each of these variables affects the probability, of having the LCV serve as a vug passageway. For both shape families (e.g., oblate and prolate ellipsoids), the vug abundance has a higher standardized β-value than either shape or size (independent variables). This result implies that, for at least the formation considered in the simulation described herein, abundance had the greatest influence on the probability of LCV being a vug passageway (e.g., the dependent variable). Vug shape was the second strongest influence on the likelihood of LCV being a vug passageway and vug size had the least weight in influencing the likelihood of LCV being a vug passageway. It is noted that while the particular example illustrated in the validations described above indicates that vug abundance had the strongest influence on the probability of an LCV being a vug passageway followed by vug shape and then vug size, it should be appreciated that the impact these different variables have with respect to influencing the likelihood of an LCV being a vug passageway may vary for different rock formations and may be detected for any particular formation using the techniques described herein.

The experiments, simulations, and validations disclosed herein indicate that the logistic regression models of the present disclosure may serve as robust estimators for the probability of a LCV being a vug passageway. The validations show that sensitivity of the logistic regression models are 96.5% for the oblate model and 100% for the prolate model. The specificity of the logistic regressions are 95%, for both the oblate and prolate models. The area under the ROC curve for observed versus predicted vug passageways is 0.98 (for prolate models) and 0.97 (for oblate models), with p-value<0.05 for both models, as described above with reference to FIG. 11. Additionally, FIG. 11 illustrates that validations comparing the observed versus predicted vug passageway in the testing data show that the ROC area under the curve for observed versus predicted vug passageways is 0.98 (for prolate models) and 0.97 (for oblate models), with p-value<0.05 for both models. Collectively, these results indicate a statistically robust predictability for use of the logistic regression models to predict performance of a reservoir in accordance with aspects of the present disclosure.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

Moreover, the scope of the present application is not intended to be limited to the particular embodiments of the process, machine, manufacture, composition of matter, means, methods and steps described in the specification.

What is claimed is:

1. A non-transitory computer-readable medium storing instructions that, when executed by one or more processors, cause the one or more processors to perform operations for evaluating permeability of a material, the operations comprising:
compiling data associated with one or more characteristics of the material;
deriving vug attribute parameters from the data;
executing a modelling engine against the vug attribute parameters;
predicting the permeability of the material based on the execution of the modelling engine against the vug attribute parameters; and
displaying information associated with the permeability of the material at a graphical user interface (GUI).

2. The non-transitory computer-readable medium of claim 1, wherein the material comprises rocks of a rock formation, and wherein predicting the permeability of the material based on the execution of the modelling engine against the vug attribute parameters comprises generating a vug connectedness probability indicative of a material permeability probability.

3. The non-transitory computer-readable medium of claim 1, wherein compiling the data associated with one or more characteristics of the material comprises receiving the data from at least one of a database and one or more sensors disposed within the material, and wherein the data comprises physical data and chemical data corresponding to the material.

4. The non-transitory computer-readable medium of claim 1, wherein the data comprises nuclear magnetic resonance (NRM) spectroscopy logs, computerized tomography (CT) logs of the material, or both.

5. The non-transitory computer-readable medium of claim 1, wherein the vug parameters comprise at least one of a mean vug diameter, a representative vug shape, a mean quantity of vugs present in the material, and a vug aspect ratio.

6. The non-transitory computer-readable medium of claim 5, wherein the representative vug shape comprises at least one of an oblate ellipsoid, a prolate ellipsoid, and a sphere, and wherein the vug aspect ratio comprises a ratio of an equatorial diameter of the representative vug shape to a polar diameter of the representative vug shape.

7. The non-transitory computer-readable medium of claim 1, wherein executing the modelling engine against the vug attribute parameters comprises performing a statistical analysis based on the vug attribute parameters.

8. The non-transitory computer-readable medium of claim 7, wherein the statistical analysis comprises a binary logistic regression algorithm, and wherein the vug attribute parameters are inputs to a binary logistic regression algorithm.

9. The non-transitory computer-readable medium of claim 8, wherein executing the modelling engine against the vug attribute parameters further comprises training the binary logistic regression equation by providing a database of vug attribute parameters as inputs to the binary logistic regression algorithm to enhance an accuracy of weights corresponding to coefficients associated with the vug attribute parameters.

10. A device for evaluating permeability of rock extracted from a rock formation, the device comprising:
a memory configured to store instructions and data associated with one or more characteristics of the rock; and
a processor configured to execute the instructions to:
derive vug attribute parameters from the data;
generate a model of vugs comprising the rock formation based, at least in part, on the vug attribute parameters; and
generate modelled rock property data that includes a probability indicative of vug connectedness based, at least in part, on the model.

11. The device of claim 10, wherein the processor is further configured to render the modelled rock property data on a graphical user interface (GUI), and wherein the device further comprises a display configured to render the GUI.

12. The device of claim 10, wherein the processor is further configured to generate secondary vug attribute parameters based on the vug attribute parameters, wherein the secondary vug attribute parameters comprises a quantity of vugs present within a rock formation, and wherein the vug attribute parameters comprises a distribution of vug diameters of vugs present within the rock formation.

13. The device of claim 12, wherein generating the model of the vugs comprises performing an ergodic analysis of the vugs based, at least in part, on the vug parameter data and on the secondary vug parameter data.

14. The device of claim 13, wherein the ergodic analysis comprises at least one of binary logistic regression, log-binomial regression, Poisson regression, Poisson regression with a robust variance estimator, and/or Cox regression.

15. The device of claim 10, wherein the probability indicative of vug connectedness indicates one of a vug passageway and an isolated vug.

16. The device of claim 10, further comprising a communication interface configured to send the modelled rock property data to a user device, and wherein the user device is configured to display the modelled rock property data to a user associated with the user device.

17. A system for evaluating permeability of rock extracted from a rock formation and for predicting a type of fluid flow through the rock formation, the system comprising:
a first device configured to generate modelled rock property data comprising a probability that the rock is permeable, the device configured to generate the modelled rock property data based on vug attribute parameters derived from data corresponding to characteristics of the rock and of the rock formation;
at least one data source of a plurality of data sources, wherein the data source is configured to provide the data to the first device; and
at least a second device configured to receive the modelled rock property data and to display the modelled rock property data.

18. The system of claim 17, wherein generating the modelled rock property data further comprises:
deriving a representative vug shape based on one of the data and the vug attribute parameters; and
using the representative vug shape to model vugs comprising the rock formation.

19. The system of claim 17, wherein the at least one data source comprises a database of characteristics corresponding to the rock and to the rock formation, and wherein at least a second data source of the plurality of data sources comprises an autonomous vehicle configured to receive the data.

20. The system of claim 19, wherein the at least the second device is further configured to:
receive the data;
partially process the data to generate partially processed data; and
send the partially processed data to the first device, wherein the first device is further configured to generate the modelled rock property data based, at least in part, on the partially processed data.
